(19) United States
(12) Patent Application Publication
(10) Pub. No.: US 2021/0145346 A1
(43) Pub. Date: May 20, 2021

(54) SYSTEMS, METHODS, AND MEDIA FOR EFFICIENT REAL-TIME EMBEDDED PROCESSING OF PHYSIOLOGICAL SIGNALS USING S TRANSFORMS

(71) Applicant: Mayo Foundation for Medical Education and Research, Rochester, MN (US)

(72) Inventors: David R. Holmes, III, Rochester, MN (US); Clifton R. Haider, Rochester, MN (US); Lukas Smital, Rochester, MN (US); Samuel Cerqueira Pinto, Rochester, MN (US); Christopher L. Felton, Rochester, MN (US)

(21) Appl. No.: 16/629,681

(22) PCT Filed: Jul. 11, 2018
(86) PCT No.: PCT/US2018/041702

§ 371 Date: Jan. 9, 2020

Related U.S. Application Data
(60) Provisional application No. 62/531,084, filed on Jul. 11, 2017.

(51) Int. Cl. A61B 5/346; G16H 50/20; A61B 5/00

(52) U.S. Cl. CPC A61B 5/346; G16H 50/20; A61B 5/7225; A61B 5/725; A61B 5/7257

(57) ABSTRACT

In accordance with some embodiments of the disclosed subject matter, mechanisms for efficient real-time embedded processing of physiological signals using S transforms are provided. In some embodiments, a system comprises: a sensor configured to monitor at least one condition of the subject and generate physiological feedback data; a processor configured to receive the physiological feedback data from the sensor and programmed to: implement a ECG Leads filter bank with a predetermined number of coefficients and taps selected to perform a Stockwell transform on the physiological feedback data and provide a frequency domain data of the physiological feedback data; analyze the frequency domain data using a physiological monitoring criteria; generate a report about the physiological condition of the subject based on the analysis of the frequency domain data using the physiological monitoring criteria; a display configured to display the report about the physiological condition of the subject.

### CROSS-REFERENCE TO RELATED APPLICATIONS

This application is based on, claims the benefit of, and claims priority to U.S. Provisional Application No. 62/531,084, filed Jul. 11, 2017, which is hereby incorporated herein by reference in its entirety for all purposes.

### STATEMENT REGARDING FEDERALLY SPONSORED RESEARCH

N/A

### BACKGROUND

In general, the ability to analyze and interpret periodic components of biomedical signals can be used to provide insights into both normal and pathophysiologic conditions. However, while advances in computing power and storage can allow remote physiological monitors to collect vast amounts of data, these devices often have small form factors and correspondingly small batteries and relatively little computing power that are inadequate for real-time analysis of the data. While time-frequency analysis is one of the most commonly used techniques in the extraction of signals from biomedical data, conventional time-frequency analysis typically requires more computing resources than are typically available in a remote monitor. Historically, such analysis of physiologic signals has been limited to in-clinic or in-hospital exams. Recently, wearable technologies and home-health monitoring have become well-accepted practices, but despite the increasing prevalence of embedded, autonomous, real-time applications, the computational efficiency of extracting signals representing clinically relevant biomarkers in frequency and time has not advanced to a point to overcome computational and power limitations constraining current wearable physiologic monitors. For example, the computational capabilities of microprocessors used to implement conventional ambulatory monitoring platforms are unable to conduct time-frequency analysis in real-time. Further, power limitations of mobile platforms do not support simultaneous collection and storage of data over long periods of time. As a result, the use of home-health monitoring technologies is constrained to minimal data collection for limited amounts of time.

Accordingly, new systems, methods, and media for efficient real-time embedded processing of physiological signals are needed.

### SUMMARY

In accordance with some embodiments of the disclosed subject matter, systems, methods, and media for efficient real-time embedded processing of physiological signals are provided that overcome the aforementioned drawbacks. In particular, the present disclosure may utilize a Stockwell transform (sometimes referred to as an S transform) for frequency analysis of time varying signals acquired by a subject. The present disclosure provides a filter system and pre-determined inputs to manage the computational burden of signal processing to achieve real-time processing and analysis, even when utilizing limited computational resources.

In accordance with some embodiments of the disclosed subject matter, a system for monitoring and providing feedback about a physiological condition of a subject is provided, the system comprising: a sensor configured to monitor at least one condition of the subject and generate physiological feedback data; a processor configured to receive the physiological feedback data from the sensor and programmed to: implement a filter bank with a predetermined number of coefficients and taps selected to perform a Stockwell transform on the physiological feedback data and provide a frequency domain data of the physiological feedback data; analyze the frequency domain data using a physiological monitoring criteria; generate a report about the physiological condition of the subject based on the analysis of the frequency domain data using the physiological monitoring criteria; a display configured to display the report about the physiological condition of the subject.

In some embodiments, the Stockwell transform is a discrete time Stockwell transform (DTST).

In some embodiments, the processor comprises a field programmable gate array, and one or more logic blocks of the field programmable gate array are configured to provide filter bank.

In some embodiments, the filter bank performs the Stockwell transform entirely in the time domain.

In accordance with some embodiments of the disclosed subject matter, a method for finding a Stockwell transform of a signal is provided, the method comprising: receiving a plurality of samples of a signal generated by a sensor, wherein the plurality of samples includes at least: i first samples of the signal from sample p−i to sample p−1, where i is an integer greater than one; a sample p of the signal; and i second samples of the signal from sample p+1 to sample p+i; providing each of the plurality of samples to a first filter comprising 2i filter taps, wherein each filter tap of the 2i filter taps of the first filter corresponds to a component of a Stockwell transform windowing function for frequency band f₀, the output of each of the 2i filter taps corresponding to a multiplication of the sample and a coefficient based on the windowing function for frequency band f₀; providing each of the plurality of samples to a corresponding filter tap of the 2i filter taps of the first filter; generating a first component of a Stockwell transform of sample p for frequency band f₀ based on a sum of the outputs of the 2i filter taps of the first filter; providing each of the plurality of samples to a second filter comprising 2i filter taps corresponding to components of a Stockwell transform windowing function for a second frequency band f₁; and generating a second component of the Stockwell transform of sample p for frequency band f₁ based on a sum of outputs of the 2i filter taps of the second filter.

In some embodiments, providing the sample to a first filter comprising 2i filter taps comprises providing the sample to one or more logic blocks of a field programmable gate array, the one or more logic blocks of the field programmable gate array are configured to provide the 2i filter taps of the first filter.

In some embodiments, the method further comprises receiving, from memory, a plurality of complex coefficients based on the windowing function for frequency band f₀, wherein each coefficient corresponds to a product of a windowing function and a Fourier kernel.

In some embodiments, the plurality of complex coefficients comprises a compact representation of the windowing function that includes only a subset of all coefficients needed to fully represent the entirety of the Fourier kernel are stored.

In some embodiments, the plurality of complex coefficients comprises a decimated subset of complex coefficients that provides an approximate representation of a full fidelity Fourier kernel of the Stockwell transform.

In some embodiments, the decimated subset is a truncation of the Fourier kernels of the Stockwell transform.

In some embodiments, the Stockwell transform for sample p and frequency band f₀ is characterized by S[p, f₀)=Σₙ₌₋ᵢⁱx[n]·w[p−n, f₀)·e^{-j2πf}^{₀}ⁿ where w[p−n, f)·e^{-j2πf}^{₀}ⁿ is the complex coefficient, and w[p−n, f) represents the windowing function and is characterized by

$$w [ p - n , {f}_{0} ) = \frac{\vert {f}_{0} \vert}{\sqrt{2 π}} \cdot {e}^{\frac{- ( {\vert {f}_{0} \vert ( p - n ) )}^{2}}{2}}$$

such that the complex coefficients for i is the complex conjugate of the complex coefficient for −i.

In some embodiments, the sensor comprises one or more electrocardiogram (ECG) leads, and wherein the signal is an ECG signal.

In some embodiments, the method further comprises calculating a physiologic feature of the sample p based on the Stockwell transform for sample p.

In some embodiments, the physiologic feature is the Shannon energy of sample p.

In some embodiments, the method further comprises providing the signal to an analog-to-digital converter that is configured to receive the signal and output the sample p at a predetermined sampling frequency.

In accordance with some embodiments of the disclosed subject matter, a system for finding an Stockwell transform of a signal is provided, the system comprising: a sensor; and a monitor comprising: a processor coupled to the sensor, the processor programmed to: receive a plurality of samples of the signal, wherein the plurality of samples includes at least: i first samples of the signal from sample p−i to sample p−1, where i is an integer greater than one; a sample p of the signal; and i second samples of the signal from sample p+1 to sample p+i; provide each of the plurality of samples to a first filter comprising 2i filter taps, wherein each filter tap of the 2i filter taps of the first filter corresponds to a component of a Stockwell transform windowing function for frequency band f₀, the output of each of the 2i filter taps corresponding to a multiplication of the sample and a coefficient based on the windowing function for frequency band f₀; provide each of the plurality of samples to a corresponding filter tap of the 2i filter taps of the first filter; generate a first component of a Stockwell transform of sample p for frequency band f₀ based on a sum of the outputs of the 2i filter taps of the first filter; provide each of the plurality of samples to a second filter comprising 2i filter taps corresponding to components of a Stockwell transform windowing function for a second frequency band f₁; and generate a second component of the Stockwell transform of sample p for frequency band f₁ based on a sum of outputs of the 2i filter taps of the second filter.

In some embodiments, the Stockwell transform is a discrete time Stockwell transform.

In some embodiments, the processor is further programmed to generate the first component of the Stockwell transform and the second component of the Stockwell transform after receiving sample p+i and prior to receiving sample p+(i+1).

In some embodiments, the system further comprises memory storing a plurality of complex coefficients based on the windowing function for frequency band f₀, wherein each coefficient corresponds to a product of a windowing function and a Fourier kernel.

In some embodiments, the processor comprises a field programmable gate array, and one or more logic blocks of the field programmable gate array are configured to provide the 2i filter taps of the first filter.

In some embodiments, the Stockwell transform for sample p has a complexity of O(NM) where N is 2i+1, and M is a number of frequency bands f₀ to fₘ analyzed to perform the Stockwell transform, wherein the system comprises one filter for each frequency band.

In some embodiments, the system further comprises an analog-to-digital converter that is configured to receive the signal and output the sample p at a predetermined sampling frequency.

In some embodiments, the sensor comprises one or more electrocardiogram (ECG) leads, and wherein the signal is an ECG signal.

In some embodiments, the processor is further configured to calculate the Shannon energy of sample p based on the Stockwell transform for sample p.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 shows an example of a system for efficient real-time embedded processing of physiological signals using S transforms in accordance with some embodiments of the disclosed subject matter.

FIG. 2 shows an example of a block diagram of a system for efficient real-time embedded processing of physiological signals using S transforms in accordance with some embodiments of the disclosed subject matter.

FIG. 3 shows an example of hardware that can be used to implement a remote monitor and a computing device of FIG. 2 in accordance with some embodiments of the disclosed subject matter.

FIG. 4 shows an example of the power spectral density of a chirp signal and results of analysis on the chirp signal carried using a short time Fourier Transform, a discrete Time S transform using a full width windowing function, and a discrete Time S transform using a truncated windowing function in accordance with some embodiments of the disclosed subject matter

FIG. 5 shows an example of a filter bank that can be used to implement efficient real-time embedded processing of physiological signals using S transforms in accordance with some embodiments of the disclosed subject matter.

FIG. 6 shows another example of hardware that can be used to implement a portion of the remote monitor of FIG. 2 in accordance with some embodiments of the disclosed subject matter.

FIG. 7 shows an example of hardware that can be used to implement efficient real-time embedded processing of physiological signals using S transforms on a special purpose processor in accordance with some embodiments of the disclosed subject matter.

FIG. 8 shows an example of a process for precomputing windowing function coefficients that can be used in a filter bank implementing a portion of a system for efficient real-time embedded processing of physiological signals using S transforms on a special purpose processor in accordance with some embodiments of the disclosed subject matter.

FIG. 9 shows an example of a process for real-time processing of a physiological signals using S transforms in accordance with some embodiments of the disclosed subject matter.

FIG. 10 shows an example of a process for real-time QRS detection from electrocardiogram signals using S transforms in accordance with some embodiments of the disclosed subject matter.

FIG. 11 shows an example of an electrocardiogram signal and an S transform representation of frequency components of the electrocardiogram signal that was generated using a system implemented in accordance with some embodiments of the disclosed subject matter.

FIG. 12 shows an example of an electrocardiogram signal and a signal indicative of the presence of a QRS complex in the electrocardiogram signal that was generated using a system implemented in accordance with some embodiments of the disclosed subject matter.

### DETAILED DESCRIPTION

In accordance with various embodiments, mechanisms (which can, for example, include systems, methods, and media) for efficient real-time embedded processing of physiological signals using S transforms are provided.

The discrete Fourier transform (DFT) is a commonly used frequency analysis approach that is used to analyze the contributions of different frequency components to a signal, which is often implemented using fast Fourier transform (FFT) techniques. However, there are several alternative approaches which have advantages and disadvantages for certain domain problems. For example, the FFT assumes that the signal being analyzed is a discrete, periodic, and infinite signal. To comply with this assumption, finite length signals are typically modeled as a repeated signal. Accordingly, for many applications of the FFT, the entire time domain signal is processed with the FFT at one time to capture as much of the signal as possible. The FFT is, however, less suitable for truly time varying signals.

In the case of time varying signals, a short-time Fourier transform (STFT) is often used as an alternative. The STFT provides an estimate of the frequency content of a signal over a fixed time window, and is computationally bounded by the use of a fixed window size. Accordingly, the selection of window size is a critical factor that determines the accuracy of the frequency analysis. For example, small windows are unable to faithfully represent slowly varying signal, while large windows limit the temporal resolution of the frequency analysis.

The S transform (sometimes referred to as the Stockwell transform) is another alternative that can be used in frequency analysis of time varying signals. The S transform balances the trade-off between the size of the time window being analyzed and frequency resolution. The S transform is often appropriate for generating a temporally local estimate of the frequency content of a signal. The S transform can be implemented as a continuous transform or as discrete transform. For example, the S transform can be implemented as a discrete time S transform (DTST) that can be computed for a given point in time p and a given frequency f:

$$S [ p , f ) = {\sum }_{n = - ∞}^{∞} x [ n ] \cdot \frac{\vert f \vert}{\sqrt{2 π}} \cdot {e}^{\frac{- ( {\vert f \vert ( p - n ) )}^{2}}{2}} {e}^{- j2 π f n} \qquad ( 1 )$$

Note that p in EQ. 1 refers to a discrete time index in the time series of n, while f is a continuous variable. Accordingly, the analysis is not limited to estimating frequency as only discrete values.

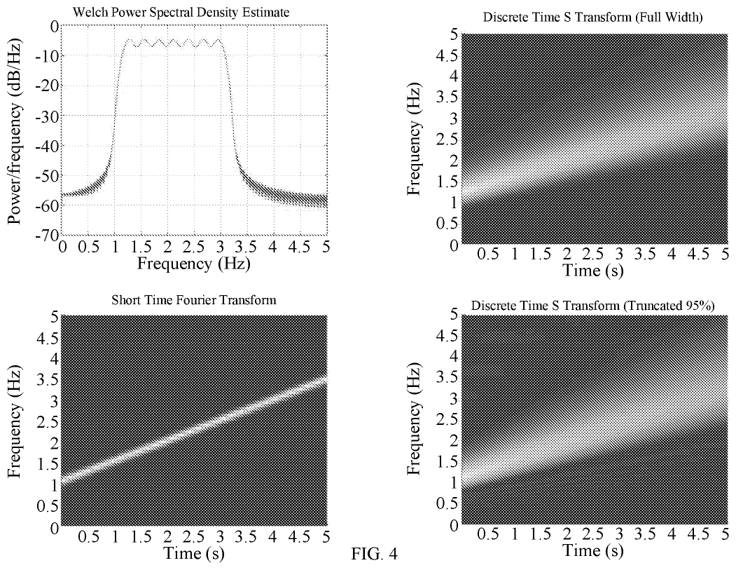

Implementation of time-frequency analysis techniques in wearable devices is challenging, as many microprocessors have neither the clock speed or memory store required to complete a frequency analysis using conventional techniques. In some embodiments, the STFT can be used to limit the amount of memory and computational cycles required to perform a real-time time-frequency analysis in a mobile device with limited computing resources. However, the STFT requires defining a fixed window which can inadequately decompose certain signals. In its original form the S transform is computationally challenging, but modifications can be made to make the S transform more suitable for embedded platforms. For example, as described below in connection with FIG. 4, the S transform can be expressed as a convolution in the time domain, making it more suitable for real-time embedded applications. As another example, the windowing function for the S transform, a frequency-dependent Gaussian, can be pre-computed for the frequency bands to be analyzed, which can reduce the amount of computations needed to perform an S transform. As yet another example, because the windowing function is a symmetric Gaussian, the number coefficients used to perform an S transform can be reduced by using each coefficient twice (e.g., once on each half of the Gaussian), which can reduce the amount of memory used when performing. As still another example, the “long tails” of the Gaussian windowing functions (i.e., where the function asymptotically approaches zero) can be truncated with little impact on the output signal, which can reduce the amount of computations (as fewer operations are required) and memory (as fewer coefficients are used) needed to perform an S transform. As a further example, the frequency bands to be analyzed can be pre-specified, and thus, can be limited to only bands of interest, which can further reduce the amount of computations and the amount of memory needed to perform an S transform.

In some embodiments of the disclosed subject matter, the S transform can be implemented such that the worst-case computational complexity of the is O(MN) where N is the window length (in discrete samples) and M is the desired frequency samples. However, due to number of zero-valued taps in the truncated windowing function (i.e., taps that can be omitted because they would contribute nothing to the final value), real-world performance can be expected to markedly exceed a technique that is O(MN) complex. In some embodiments, a filter bank which can be tuned to a specific application (e.g., ECG processing) can be used to implement a computationally efficient S transform.

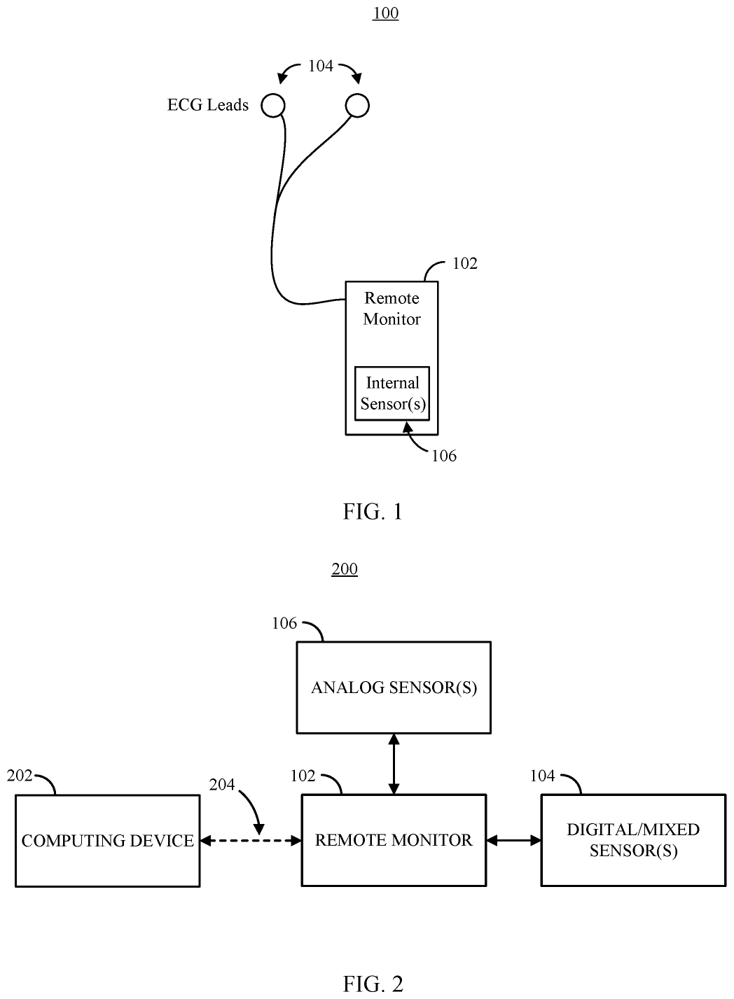

FIG. 1 shows an example 100 of a system for efficient real-time embedded processing of physiological signals using S transforms in accordance with some embodiments of the disclosed subject matter. As shown in FIG. 1, system 100 can include a remote physiological monitor 102 configured to sense and record signals from one or more electrocardiogram (ECG) leads 104 (or other external sensors), and/or one or more internal sensors 106 (e.g., motion sensors for measuring gross motor activity). In some embodiments, one or more sensors (e.g., ECG lead 104) can be exterior to remote monitor 102, and one or more sensors (e.g., internal sensor 106) can be integrated into remote monitor 102. In some embodiments, remote monitor 102 can be configured to be worn by a subject to be monitored (e.g., within a pocket of a tight fitting garment) such that information about the subject can be continuously collected. Note that although only ECG leads are shown in FIG. 1, the mechanisms described herein can be useful with any techniques that capture data that can be analyzed using frequency analysis techniques. For example, the mechanisms described herein can be used to analyze information gathered using electroencephalography (EEG) techniques, photoplethysmography (PPG) techniques, magnetoencephalography (MEG) techniques, electromyogram (EMG) techniques, etc.

FIG. 2 shows an example 200 of a block diagram of a system for efficient real-time embedded processing of physiological signals using S transforms in accordance with some embodiments of the disclosed subject matter. As shown in FIG. 2, in some embodiments, remote monitor 102 can be coupled to any suitable number of digital and/or mixed sensors (e.g., sensors that provide a digital signal indicative of an output, but which may internally produce analog signals, such as a motion sensor), and/or any suitable number of analog sensors (e.g., sensors that provide an analog signal, such as ECG leads 104). Additionally, in some embodiments, remote monitor 102 can communicate with a computing device 202 via a communication link 204 using any suitable technique or combination of techniques. For example, communication link 204 can be a wired or wireless link through which remote monitor 102 can provide recorded data (e.g., reflecting an analysis of frequency components generated using the mechanisms described herein).

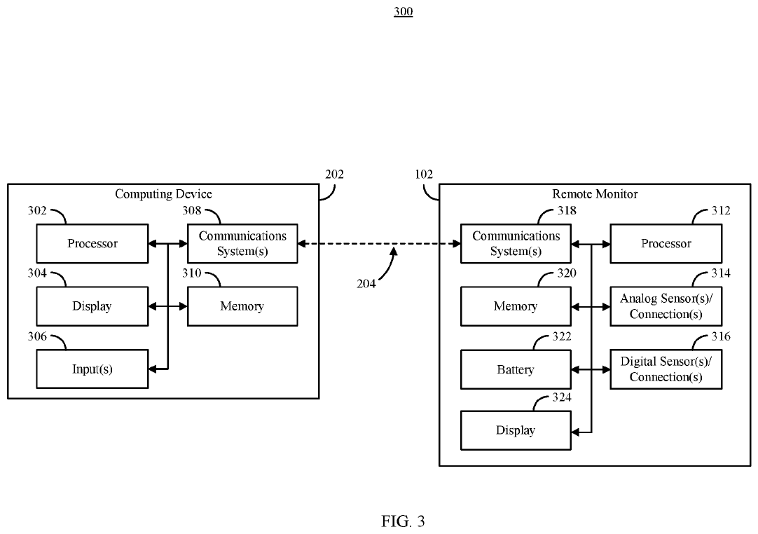

FIG. 3 shows an example 300 of hardware that can be used to implement remote monitor 102 and computing device 202 of FIG. 2 in accordance with some embodiments of the disclosed subject matter. As shown in FIG. 3, in some embodiments, remote monitor 102 can include a processor 312, one or more analog sensors 314 (and/or hardware configured to receive signals from one or more analog sensors), one or more digital sensors and/or mixed sensors 316 (and/or hardware configured to receive signals from one or more digital or mixed sensors), one or more communication systems 318, memory 320, a battery (and/or other power source) 322, and/or a display 324.

In some embodiments, processor 312 can be any suitable hardware processor or combination of processors, such as a central processing unit (CPU), a graphics processing unit (GPU), a microcontroller (MCU), a field programmable gate array (FPGA), an application-specific integrated circuit (ASIC), etc. In some embodiments, communications system(s) 318 can include any suitable hardware, firmware, and/or software for communicating information to computing device 202, over communication link 204, over any other suitable communication link or combination of communication links, and/or over any suitable communication network or combination of networks. For example, communications system(s) 318 can include one or more transceivers, one or more communication chips and/or chip sets, etc. In a more particular example, communications system(s) 318 can include hardware, firmware and/or software that can be used to communicate data over a coaxial cable, a fiber optic cable, an Ethernet connection, a USB connection, to establish a Wi-Fi connection, a Bluetooth connection, a cellular connection, etc.

In some embodiments, memory 320 can include any suitable storage device or devices that can be used to store instructions, values, etc., that can be used, for example, by processor 312 to receive signals via analog sensors 314 and/or digital sensors 316, to perform an S transform based on signals received via analog sensors 314 and/or digital sensors 316, to store S transform data in memory 320, and/or transmit S transform data to computing device 202 via communications system(s) 318, etc. Memory 312 can include any suitable volatile memory, non-volatile memory, storage, or any suitable combination thereof. For example, memory 310 can include RAM, ROM, EEPROM, one or more flash drives, one or more hard disks, one or more solid state drives, one or more optical drives, etc. In some embodiments, memory 320 can have encoded thereon a program for controlling operation of processor 312 to perform an S transform based on physiological and/or environmental signals. In some such embodiments, processor 312 can execute at least a portion of the program to execute at least a portion of processes 800, 900, and/or 1000 as described below in connection with FIGS. 8-10, respectively.

In some embodiments, display 324 of remote monitor 102 can be any suitable device for outputting information, such as a liquid crystal display, a segmented liquid crystal display, a light-emitting diode (LED) display (e.g., an organic LED/OLED display) a speaker, etc. Note that, although not shown in FIG. 3, remote monitor 102 can include one or more input devices, such as one or more hardware buttons, one or more software buttons, a touchscreen, a microphone, etc., for receiving user input and/or a display, a speaker, etc., for outputting information for consumption by a user.

In some embodiments, computing device 202 can include a processor 302, a display 304, one or more inputs 306, one or more communication system(s) 308, and/or memory 310. In some embodiments, processor 302 can be any suitable hardware processor or combination of processors, such as a CPU, a GPU, MCU, FPGA, ASIC, etc. In some embodiments, display 304 can include any suitable display devices, such as a computer monitor, a touchscreen, a television, etc. In some embodiments, inputs 306 can include any suitable input devices and/or sensors that can be used to receive user input, such as a keyboard, a mouse, a touchscreen, a microphone, etc.

In some embodiments, communications system(s) 308 can include any suitable hardware, firmware, and/or software for communicating with remote monitor 102, for communicating information over communication link 204, and/or for communicating over any other suitable communication links and/or communication networks. For example, communications system(s) 308 can include one or more transceivers, one or more communication chips and/or chip sets, etc. In a more particular example, communications systems 308 can include hardware, firmware and/or software that can be used to establish a coaxial connection, a fiber optic connection, an Ethernet connection, a USB connection, a Wi-Fi connection, a Bluetooth connection, a cellular connection, etc.

In some embodiments, memory 310 can include any suitable storage device or devices that can be used to store instructions, values, etc., that can be used, for example, by processor 302 to present content using display 304, to communicate with one or remote monitors (e.g., remote monitor 102). Memory 310 can include any suitable volatile memory, non-volatile memory, storage, or any suitable combination thereof. For example, memory 310 can include RAM, ROM, EEPROM, one or more flash drives, one or more hard disks, one or more solid state drives, one or more optical drives, etc. In some embodiments, memory 310 can have encoded thereon a computer program for controlling operation of processor 302. In some such embodiments, processor 302 can execute at least a portion of the computer program to receive signals, S transform data, and/or analysis based on S transform data from remote monitor 102, to present information based on the signals, S transform data, and/or analysis based on S transform data, etc. In some embodiments, processor 302 can execute one or more portions of processes 800, 900, and/or 1000 described below in connection with FIGS. 8-10. In some embodiments, computing device 202 can be any suitable computing device or combination of computing devices, such as a personal computer, a laptop computer, a tablet computer, a smartphone, a wearable computer, a server, etc.

Although not shown, in some embodiments, communication link 204 can include multiple communication links that form a portion of any suitable communication network or combination of communication networks. For example, communication link 204 can be a combination of one or more links in a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, etc.

FIG. 4 shows an example of the power spectral density of a chirp signal and results of analysis on the chirp signal carried using a short time Fourier Transform, a discrete Time S transform using a full width windowing function, and a discrete Time S transform using a truncated windowing function in accordance with some embodiments of the disclosed subject matter. As shown in FIG. 4, much like other frequency analysis techniques, the DTST can be used to filter the time domain signal shown in the upper left of FIG. 4 with specialized windowing functions to estimate frequency content. However, in contrast to the STFT, the local estimate of frequency content is not based on a fixed window size. Rather, the window size can vary based on the frequency estimate that is desired. For example, EQ. (1) can be decomposed into the following two parts:

$$\begin{matrix}S [ p , f ) = {\sum }_{n = - ∞}^{∞} x [ n ] \cdot w [ p - n , f ) \cdot {e}^{- j2 π f n} & ( 2 ) \\ w [ p - n , f ) = \frac{\vert f \vert}{\sqrt{2 π}} \cdot {e}^{\frac{- ( {\vert f \vert ( p - n ) )}^{2}}{2}} & ( 3 )\end{matrix}$$

In this form, w[p−n, f) can represent a windowing function applied to the data during the computation of the S transform. Note that as used herein, f is generally a value in hertz, but this is merely an example and any unit of periodicity can be used. Note that the windowing function is frequency dependent such that, in the case of low frequency components (e.g., frequency components that vary more slowly in time) the DTST has lower time domain resolution and higher frequency resolution. Whereas, in the case of high frequency content the DTST has higher time domain resolution and lower frequency resolution. This can be observed in the analyses of an example synthetic signal shown in FIG. 4. Using Matlab 2016a, a chirp was constructed spanning from 1 Hz to 3 Hz over a 5 second window. The upper left of FIG. 4 shows a power spectral density plot (more particularly, a Welch power spectral density) of the chirp signal. The lower left of FIG. 4 shows a STFT of the chirp signal, which decodes the time-frequency signal with relatively low resolution in the frequency domain. A DTST using a full width windowing function is shown in the upper right of FIG. 4, which shows relatively high time resolution at the cost of lower frequency resolution for higher frequency components. As shown in the lower right of FIG. 4, a DTST using a windowing function truncated to maintain 97% of the area under the Gaussian without a significant impact on the result of the S transform.

While FIG. 4 shows that the DTST has advantages when compared to the STFT in certain applications, there are some disadvantages for conventional DTST, especially for embedded applications when computing resources and/or power is limited. For example, the computational complexity of a naive implementation of DTST is O(N³) with the possibility of achieving O(N² log N) using some optimizations. An implementation (Fast DST) having a complexity of O(N log N) has been proposed (see, e.g., R. A. Brown et al., “A Fast Discrete S-transform for biomedical signal processing,” IEEE, 2008), which includes pre-computing the windowing functions and using a specific sampling scheme that efficiently computes the time-frequency subspaces. This Fast DST technique uses a dyadic sampling strategy in combination with downsampling to reduce the computational burden, and three values are pre-computed—the Fourier transform of the signal, the windowing function, and the complex kernel values. Once computed, the technique loops through each region of time-frequency space. For each block, the Fourier transform of the signal is band-passed, and the inverse Fourier transform is computed, which is then multiplied with the windowing and kernel values in order to calculate the positive and negative frequency values for each block.

As described above, the Fast DST requires both the Fourier transform and inverse Fourier Transform of the signal. In some embodiments, the mechanisms described herein can perform an S transform entirely in the time domain by taking advantage of the convolution theorem, the entire computation can occur in the time domain. In order to ensure that the system is causal, incoming data can be delayed to shift the computation. For example, rather than performing an S transform for a most recently received sample, the S transform can be performed on a sample p received n samples before the most recent sample, and the n samples received before and after sample p can be used in the S transform. Additionally, in some embodiments, the windowing function (e.g., the Gaussian shown in EQ. (3)) can be pre-computed for a given set of frequencies. Further reductions in computational resources used to perform the S transform can be realized, in some embodiments, by truncating the windowing function from the function in EQ. (3) having infinite support, without significantly impacting the computation. In some embodiments, the truncated windowing function and the Fourier kernel values (i.e., e^{-j2πfn}) can be pre-multiplied and stored as complex coefficients. Note that, because the complex windowing function is symmetric, in some embodiments, these complex coefficients can be stored using only ½ of the taps that would otherwise be used if all of the coefficients were calculated. For example, the coefficients for each positive value of n can be calculated by multiplication of the windowing function and the Fourier kernel and stored (e.g., using a register), and that coefficient can be used during calculation of the S transform for both sides of the windowing function (i.e., once for the +n side, and once for the −n side).

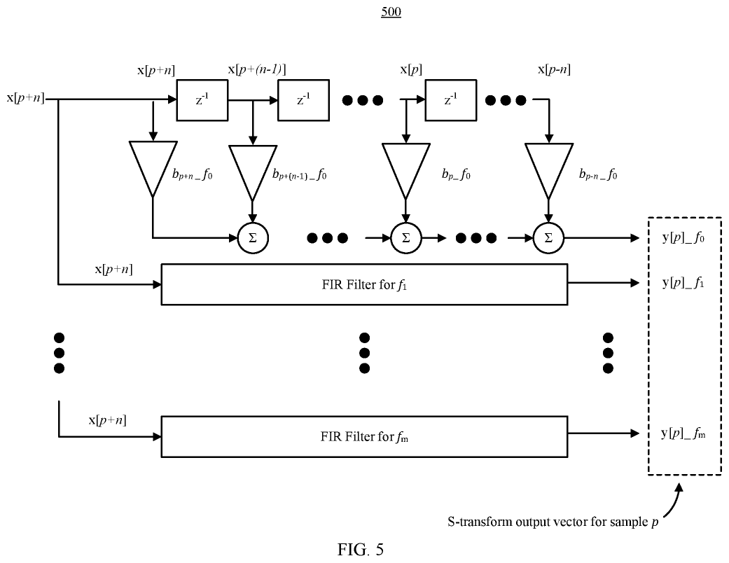

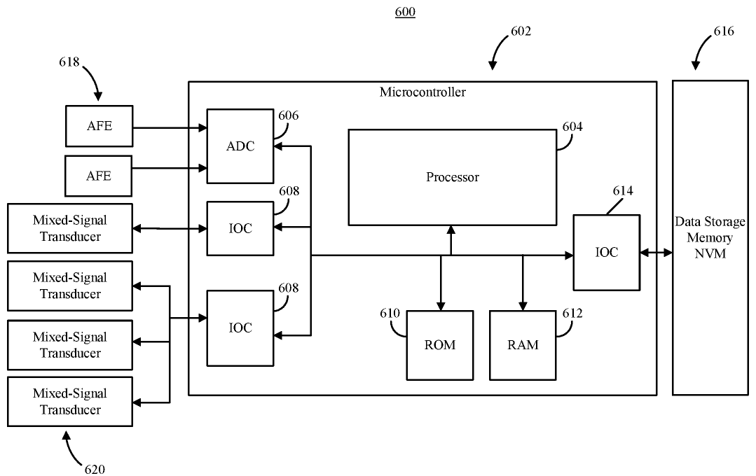

FIG. 5 shows an example 500 of a filter bank that can be used to implement efficient real-time embedded processing of physiological signals using S transforms in accordance with some embodiments of the disclosed subject matter. As shown in FIG. 5, filter bank 500 can receive, at a time p+n, a sample x[p+n] of a signal from a sensor (e.g., ECG leads 104). In some embodiments, sample x[p+n] can be distributed to m finite impulse response (FIR) digital filters for m frequency bands. For example, sample x[p+n] can be provided as input to a FIR corresponding to frequency band f₀ with taps corresponding to the windowing function of the S-transform. In a more particular example, as shown in FIG. 5, in some embodiments, sample x [p+n] can be passed to a next tap via a unit delay component (denoted in z-transform notation as z⁻¹) and multiplied by a complex coefficient

$${b}_{{[ p + n ]}_{{f}_{0}}}$$

that has been pre-computed and stored based on EQ. (2) and (3). For example, coefficient

$${b}_{{[ p + n ]}_{{f}_{0}}}$$

can be calculated by multiplying the windowing function for sample p+n at the center frequency f₀ of the frequency band

$$( i . e . , w [ p + n , {f}_{0} ) = \frac{\vert {f}_{0} \vert}{\sqrt{2 π}} \cdot {e}^{\frac{- ( {\vert {f}_{0} \vert ( p - n ) )}^{2}}{2}} ,$$

and the corresponding Fourier kernel (i.e., e^{-j2πf}^{₀}ⁿ). Similarly, the previous sample x[p+(n−1)] can be multiplied by a complex coefficient

$${b}_{{[ p + ( n - 1 ) ]}_{{f}_{0}}}$$

that is based on the windowing function for sample p+(n−1) at frequency f₀. This can be repeated for each frequency band from p+n to p−n, including for sample p (for which the complex coefficient is

$$\frac{\vert {f}_{0} \vert}{\sqrt{2 π}} , as n = 0 ) .$$

The output y[p]_{f}_{₀} of the summation for frequency band f₀ can represent the contribution of that frequency to sample p. Similar filters can be implemented for each frequency band, resulting in a vector y[p] representing the contributions of each frequency band to the value of sample p, which represents the result of the DTST. As described above, the number of taps can be set to preserve 97% of the area under the Gaussian by selecting n based on the frequency at which the signal is samples (e.g., for higher sampling rates, n can be set higher, and vice versa). Note that this is merely an example, and the windowing functions can be truncated at any suitable value that preserves enough information to perform a reliable analysis.

Additionally, as described above in connection with FIG. 4, because the windowing function is symmetric, the complex coefficients on each side of the sample being analyzed are complex conjugates. For example, referring to FIG. 5,

$${b}_{{[ p + n ]}_{{f}_{0}}}$$

is the complex conjugate of

$${b}_{{[ p - n ]}_{{f}_{0}}} .$$

Accordingly, as described above in connection with FIG. 4, the amount of coefficients to be stored can be reduced by about half by leveraging this symmetry.

An example system was implemented for use with ECG detection, with window functions calculated for 10 Hz to 25 Hz in steps of 1 Hz using Matlab 2016a. Note that this is merely an example, and the distance between frequency bands can be adjusted based on the application and frequency range. As described above, in order to reduce the number of filter coefficients, the filter taps were truncated to preserve 97% of the area under the Gaussian. In order to map the techniques described herein onto a microcontroller, the filter coefficients were transformed into fixed point values and stored as a 16-bit constant array. A simulated ECG signal was sampled (e.g., at 360 Hz), and an S-transform was performed for each sample (with a delay of about 100 samples to insure that an adequate amount of the signal was being analyzed). The output of the S transform was used to calculate Shannon energy of each sample from 10 Hz to 25 Hz, and the Shannon energy of each sample was used to detect QRS complexes in the ECG signal, based on techniques described in L. Smital et al., “Towards Real-Time QRS Feature Extraction for Wearable Monitors,” 2016 IEEE EMBC, which is hereby incorporated by reference herein in its entirety). The techniques were implemented in C and loaded onto custom wearable devices containing an MSP430 F5528 using IAR Embedded Workbench, and an ECG simulator was attached to a 12-bit analog-to-digital converter (ADC) coupled to the microprocessor.

FIG. 6 shows an example 600 of hardware that can be used to implement a portion of remote monitor 102 of FIG. 2 in accordance with some embodiments of the disclosed subject matter. Remote physiological monitors are generally configured to be incorporated into a minimal form factor and to achieve low power usage over long run-times. These multiple constraints (which can, for example, limit the battery size) can require tradeoffs when designing a wearable device.

As shown in FIG. 6, a low-power remote monitor and recorder can include a microcontroller 602, which can include a processor 604, an ADC 606, one or more I/O pins 608 (e.g., configured as interrupt on change (IOC) I/O pins), read only memory 610, random access memory 612, one or more pins 614 configured to connect MCU 602 to non-volatile memory 616. In some embodiments, MCU 602 can receive signals from one or more analog front ends (AFEs) 618 and/or mixed-signal transducers 620. In such embodiments, CPU 604 can use a program (e.g., a program that implements one or more of portions of processes 800, 900, and/or 1000 described below in connection with FIGS. 8-10) and/or values (e.g., complex coefficients for implementing filter bank 500 described above in connection with FIG. 5) stored by ROM 610 to perform an S transform on signals received via ADC 606 and/or I/O pins 608. Additionally, in some embodiments, during operation the program and/or values can be stored in ROM 410 and/or RAM 412 during operation.

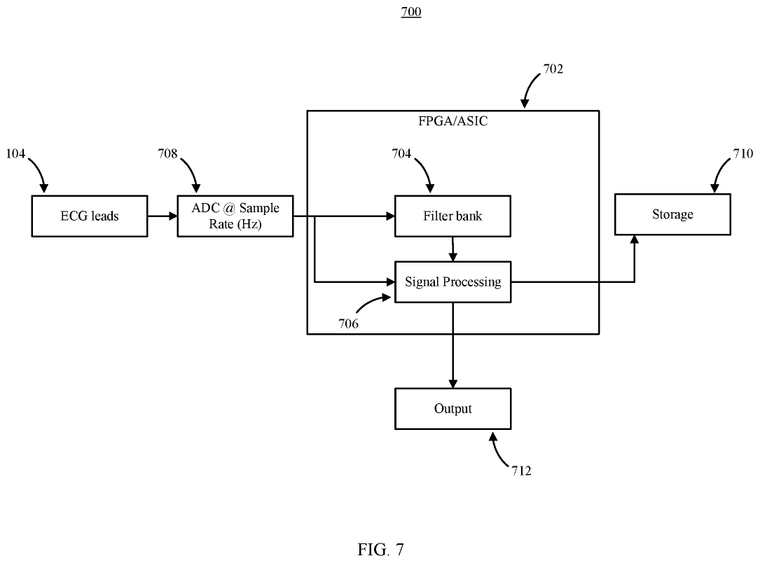

Alternatively, for example as described below in connection with FIG. 7, in some embodiments, processor 604 can be implemented using a special purpose processor such as an FPGA or ASIC, and filter bank 500 and/or coefficients for filter bank 500 can be implemented, at least in part, using hardware components such as logic gates, rather than being implemented entirely as software. For example, logic blocks of an FPGA can be configured to implement one or more portions of filter bank 500. As another example, an ASIC can be configured to implement one or more portions of filter bank 500.

FIG. 7 shows an example 700 of hardware that can be used to implement efficient real-time embedded processing of physiological signals using S transforms on a special purpose processor in accordance with some embodiments of the disclosed subject matter. As shown in FIG. 7, a low-power remote monitor and recorder can include a special purpose processor 702, such as an FPGA or an ASIC, that can be configured to implement a filter bank 704 and signal processing components 706. For example, filter bank 704 can be an implementation of filter bank 500 described above in connection with FIG. 5 that is implemented at least in part by interconnections between logic gates (i.e., rather than being implemented in software). As another example, signal processing components 706 can be configured to perform one or more signal processing operations. In a more particular example, signal processing components 706 can be configured to analyze the frequency components of a signal based on the output of the filter bank. As another example, signal processing components 706 can be configured to encode the received signal (e.g., received from ECG leads 104) for storage and/or output. In some embodiments, special purpose processor 702 can include RAM, ROM (e.g., implemented with one or more memory blocks), first-in-first-out buffers, etc.

In some embodiments, special purpose processor 702 can receive the signal from one or more analog to digital converts (ADC) 708 sampling a signal at a particular sample rate. For example, ADC 708 can sample a signal from ECG leads 104 at a sample rate suitable to capture information in the signal at a resolution that facilitates time frequency analysis of the signal. In a more particular example, ADC 708 can sample the signal from ECG leads 104 at a rate between 250 and 500 hertz (Hz). In another more particular example, ADC 708 can sample the signal from ECG leads 104 at 360 hertz, 400 Hz, etc.

In some embodiments, signal processing components 706 can provide the signal and/or the results of one or more analyses to storage 710 (e.g., non-volatile memory 616, memory 320, etc.). Additionally or alternatively, in some embodiments, signal processing components 706 can provide the signal, the results of one or more analyses to an output 712, and/or information indicative of the signal and/or results of one or more analyses. For example, output 712 can be a communication system (e.g., communication system 318) to transfer the information to another device (e.g., via a Bluetooth or other wireless connection, via a wired connection, etc.). As another example, output 712 can be a user interface (e.g., a display or other visual interface such as an indicator LED, an audio interface such as a speaker, a haptic interface, etc.), that can present information related to the signal (e.g., current pulse rate, a running average pulse rate over a predetermined period of time, etc.) and/or analysis (e.g., a detection of arrhythmia, etc.).

Note that using a special purpose processor to implement subject matter described herein can reduce the amount of power used to perform an analysis, potential extending the amount of data that can be collected and/or analyzed, facilitating complex processing of the data using relatively little power (e.g., compared to a general purpose processor performing the same operations). However, implementing subject matter described herein can limit the flexibility of the device in which it is implemented, for example, because the filter bank is generally configured to operate best at a particular sampling frequency (e.g., 360 Hz, 400 Hz, etc.). This can make it more difficult to adjust analysis performed by the device in real time (e.g., it may require reprogramming the processor, which is generally more difficult than updating or changing software).

FIG. 8 shows an example 800 of a process for precomputing windowing function coefficients that can be used in a filter bank implementing a portion of a system for efficient real-time embedded processing of physiological signals using S transforms on a special purpose processor in accordance with some embodiments of the disclosed subject matter. As shown in FIG. 8, process 800 can begin at 802 by subdividing a range of signal frequencies to be analyzed into m frequency bands fᵢ at a resolution adequate to perform one or more time-frequency analyses. For example, in the case of ECG, frequencies in the range of 10 Hz to 25 Hz can be used to detect QRS complexes in the ECG signal with greater than 99% accuracy. In such an example, process 800 can include subdividing the 10 Hz to 25 Hz range into 15 bands with a resolution of about 1 Hz.

At 804, process 800 can compute, for each frequency band fᵢ, 2n+1 window function values to use to determine the S transform for that frequency band. Process 800 can use any suitable technique or combination of techniques to compute the 2n+1 window function values, such as based on EQ. (3) described above in connection with FIG. 4. As described above in connection with FIG. 5, n can be the number of samples received before and after a sample p being analyzed that are used to determine the frequency components of the sample. Alternatively, in some embodiments, process 800 can compute n+1 window function values, as the window function may be symmetric with the values for samples received before sample p being the same as the values for samples received after sample p.

At 806, process 800 can compute, for each frequency band fᵢ, 2n+1 phase factor values to use to determine the S transform for that frequency band. Process 800 can use any suitable technique or combination of techniques to compute the 2n+1 phase factor values, such as based on EQ. (2) described above in connection with FIG. 4. Alternatively, in some embodiments, process 800 can compute n phase factor values, as the phase factor may be symmetric with the values for samples received before sample p being the complex conjugate of the corresponding values for samples received after sample p as described above in connection with FIG. 4.

At 808, process 800 can compute, for each frequency band fᵢ, 2n+1 complex coefficient values to apply to samples from a signal to determine the S transform for that frequency band by multiplying the corresponding window function and phase factor. Alternatively, in some embodiments, process 800 can compute n complex coefficient values, as the complex coefficients may be symmetric around sample p, as described above in connection with FIG. 4.

At 810, process 800 can store the compute complex coefficients for use as coefficients in a filter bank implementing the S transform for the selected frequency range. In some embodiments, the coefficients can be stored as fixed point values and stored as a 16-bit constant array for use by a processor being used to implement the filter bank. For example, the coefficients can be used when programming an FPGA to implement a filter bank to execute the S transform in logic blocks, when designing an ASIC to implement a filter bank to execute the S transform in hardware, when programming a general purpose processor to implement a filter bank to execute the S transform in software, etc.

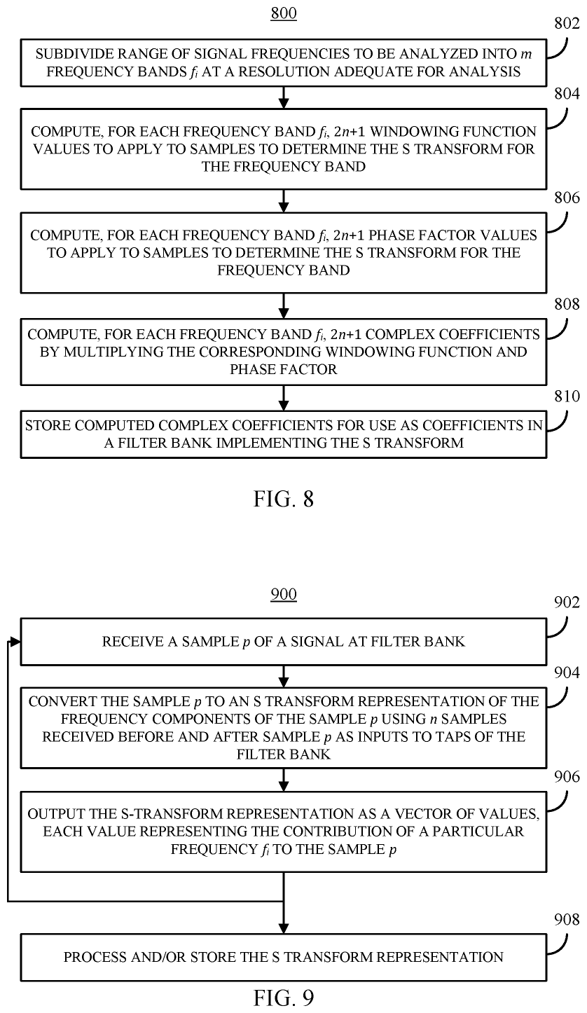

FIG. 9 shows an example 900 of a process for real-time processing of a physiological signals using S transforms in accordance with some embodiments of the disclosed subject matter. As shown in FIG. 9, process 900 can begin at 902 by receiving a sample p of a signal at a filter bank, such as filter bank 500 described above in connection with FIG. 5.

At 904, process 900 can convert the sample p into an S transform representation of the frequency components of the sample p using n samples received before and after sample p as inputs to taps of the filter bank. For example, as described above, the S transform analysis for sample p can be performed upon receiving sample p+n at the filter bank.

At 906, process 900 can output the S transform representation as a vector of values with each value representing the contribution of a particular frequency band fᵢ to the value of sample p. For example, as described above in connection with FIG. 5, the filter bank can have m outputs that each represent the contribution of a particular frequency band.

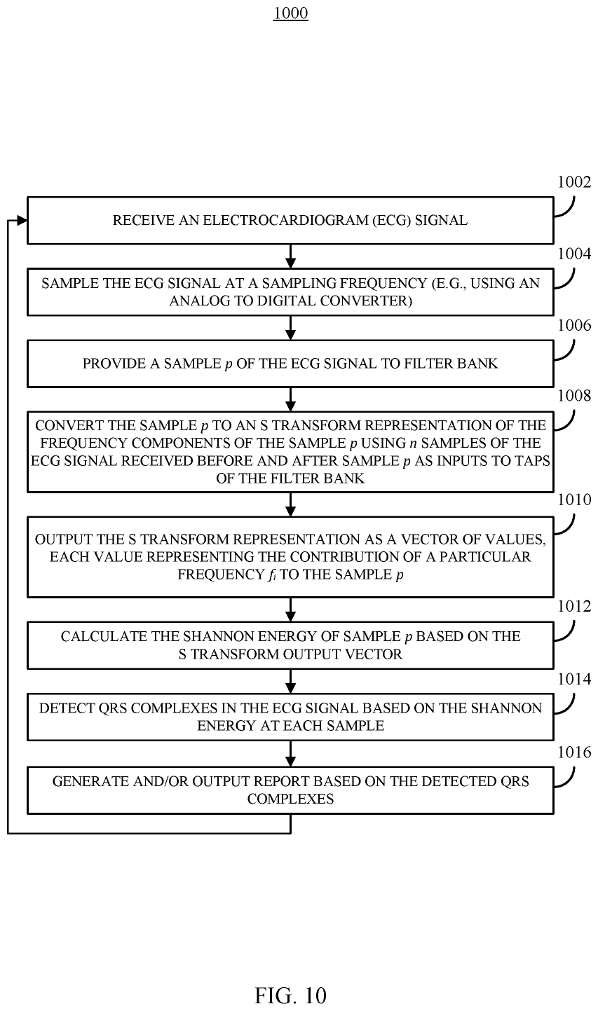

At 908, process 900 can process and/or store the S transform representation output at 906. For example, process 900 can associate the S transform representation with sample p in memory (e.g., using an index number of sample p). As another example, process 900 can use the S transform representation in a further analysis using a physiological monitoring criteria. In a more particular example, as described below in connection with FIG. 10, process 900 can use the S transform representation for QRS detection in an ECG signal. Additionally, in some embodiments, process 900 can generate and/or present a report about the physiological condition of a subject based on the S transform representation output at 906. For example, as described below in connection with FIG. 10, the report can include a pulse rate, an indication of whether an arrhythmia event has been detected and/or the frequency, number, etc., of arrhythmia events that have occurred (e.g., in the past hour, day, etc.). In some embodiments, process 900 can return to 902 to receive a new sample when the S transform for the previous sample is being processed and/or stored.

FIG. 10 shows an example 1000 of a process for real-time QRS detection from electrocardiogram signals using S transforms in accordance with some embodiments of the disclosed subject matter. As shown in FIG. 10, process 1000 can receive an ECG signal at 1002 from any suitable source. For example, process 1000 can receive the ECG signal from ECG leads 104.

At 1004, process 1000 can sample the ECG signal at a predetermined sampling frequency to create a digital signal at the sampling frequency. In some embodiments, process 1000 can use any suitable technique or combination of techniques to sample the ECG signal. For example, in some embodiments, process 1000 can sample the ECG signal using an ADC operating at the sampling frequency. As another example, process 1000 can sample the ECG signal using multiple ADCs operating at the sampling frequency that each receive a signal from a particular frequency band (e.g., frequency band fᵢ). In some embodiments, the ECG signal can be sampled at any suitable frequency, such as a rate between 250 and 500 Hz. In a more particular example, the ECG signal can be sampled at 360 hertz, 400 Hz, etc.

At 1006, process 1000 can provide a sample p to a filter bank configured to perform an S transform of the sample. In some embodiments, process 1000 can provide sample p to the filter bank from an ADC, and the filter bank can act as a bank of band pass filter to subdivide the signal into frequency components.

At 1008, process 1000 can convert the sample p to an S transform representation of the frequency components of the sample p using the filter bank. In some embodiments, process 1000 can use any suitable number of additional samples received before and after sample p when finding the S transform of sample p. For example, as described above in connection with FIG. 5, process 1000 can use n samples received prior to sample p and n samples received after sample p to determine the S transform for sample p. Accordingly, when process 1000 is receiving sample p at 1006, process 1000 can be finding the S transform for sample [p−n] using samples [p−2n] to p, and process 1000 can use samples [p−n] to [p+n] when finding the S transform for sample p. In light of this, process 1000 can receive sample [p+(n+1)] while finding the S transform for sample p.

At 1010, process 1000 can output the S transform representation as a vector of values with each value representing the contribution of a particular frequency band fᵢ to the value of sample p. For example, as described above in connection with FIG. 5, the filter bank can have m outputs that each represent the contribution of a particular frequency band.

At 1012, process 1000 can include calculating the Shannon energy of the ECG signal at sample p using any suitable technique or combination of techniques. For example, techniques described in L. Smital et al., “Towards Real-Time QRS Feature Extraction for Wearable Monitors,” 2016 IEEE EMBC can be used to calculate the Shannon energy from the S transform. As another example, techniques described in Z. Zidelmel et al., “QRS detection using S-Transform and Shannon Energy,” Computer methods and programs in biomedicine, vol. 116, pp. 1-9, 2014, which is hereby incorporated by reference herein in its entirety can be used to calculate the Shannon energy from the S transform.

At 1014, process 1000 can detect QRS complexes in the ECG signal based on the Shannon energy at each sample using any suitable technique or combination of techniques. For example, techniques described in L. Smital et al., “Towards Real-Time QRS Feature Extraction for Wearable Monitors,” can be used to detect QRS complexes in the ECG signal based on the Shannon energy for each sample. As another example, techniques described in Z. Zidelmel et al., “QRS detection using S-Transform and Shannon Energy,” Computer methods and programs in biomedicine, vol. 116, pp. 1-9, 2014, which is hereby incorporated by reference herein in its entirety can be used to calculate the Shannon energy from the S transform.

In some embodiments, process 1000 can return to 1002 to continue to receive ECG signals while detecting QRS complexes in the ECG signal based on the Shannon energy of each sample. Note that, because n samples after the sample p being evaluated are used to determine the S transform, the detection of each QRS complex is delayed by at least n/sampling rate seconds. For example, if n is 100 (meaning that information from 201 samples, including sample p, are used when calculating the S transform of sample p) and the sampling rate is 400 Hz, then the S transform cannot be calculated before 100 samples are received after sample p, which corresponds to at least about 0.25 seconds.

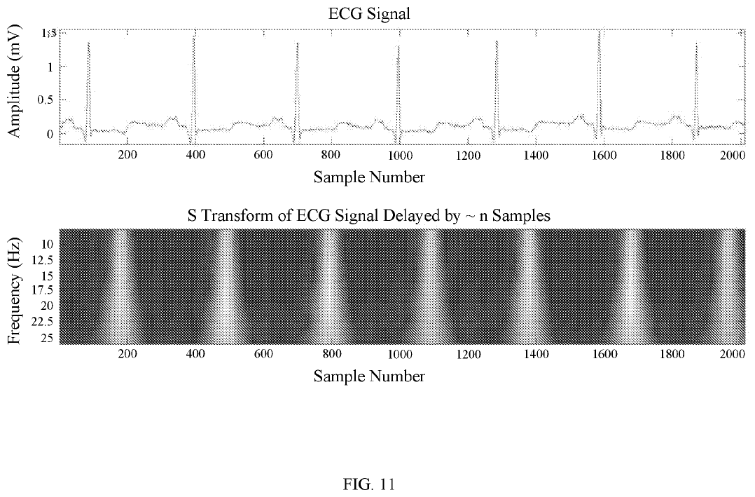

At 1016, process 1000 can generate and/or output a report based on the detected QRS complexes. For example, in some embodiments, process 1000 can determine and/or output a pulse rate based on the detected QRS complexes. As another example, process 1000 can detect and/or alert a user to one or more arrhythmia events based on the QRS complexes, etc. FIG. 11 shows an example of an electrocardiogram signal and an S transform representation of frequency components of the electrocardiogram signal that was generated using a system implemented in accordance with some embodiments of the disclosed subject matter. As shown in FIG. 11, the S transform at each frequency (from about 10 Hz to about 25 Hz) is a maximum about 100 samples after the QRS complex is observed in the ECG signal. This corresponds to roughly the number of samples captured after the QRS complex in the ECG that are used to calculate the S transform for samples corresponding to the QRS complex.

The filter bank used to implement the system that generated the S transforms of FIG. 11 included 3116 taps across 15, using 1558 coefficients with the number of taps truncated to maintain 97% of the area under the Gaussian. This resulted in 3116 real multiplications, and 3116 real adds per input evaluation.

The results shown in FIG. 11 were generated by implementing the techniques described herein in C and loading the software onto custom wearable devices containing an MSP430 F5528 using IAR Embedded Workbench, and an ECG simulator was attached to a 12-bit analog-to-digital converter (ADC) coupled to the microprocessor using a data sampling set to 360 Hz and 400 Hz. The delay introduced into the acquisition between when a sample was acquired and when it's S transform was calculated was 0.22 seconds for data sampled at 360 Hz and 0.2 seconds for data sampled at 400 Hz. The execution time required to perform the S transform for a particular sample was measured via an instruction-set-simulator that was used to count clock cycles required to execute the compiled code, and is shown in TABLE 1.

TABLE 1

| Sample Rate | | | |
|---|---|---|---|
| [Hz] | Time [ms] | Clock Cycles | Utilization |
| 360 | 12.1 | 96,972 | 43.64% |
| 400 | 13.4 | 107,050 | 53.53% |

Power measurements taken from the device showed a 1.8 milliamp (mA) or 2.0 mA current draw for a sampling rate of 360 Hz and 400 Hz, respectively. The baseline performance of the device without QRS detection was 1.0 mA. Although this is roughly a doubling of the current draw, it is still sufficiently efficient to capture and analyze data for several days. Device performance can be further tuned by varying truncation of the windowing function and adjustment of the number of frequency bands being analyzed. Additionally, implementation using a special purpose processor (e.g., an FPGA or ASIC) can further reduce power consumption by reducing excess computational resources.

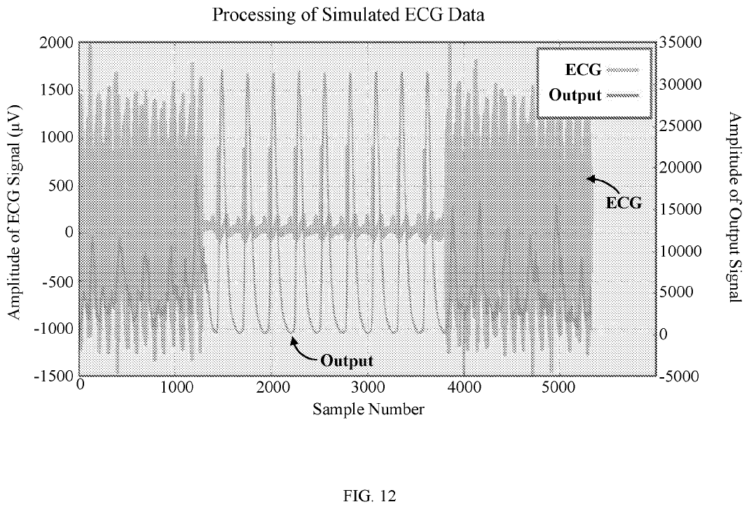

FIG. 12 shows an example 1200 of an electrocardiogram signal and a signal indicative of the presence of a QRS complex in the electrocardiogram signal that was generated using a system implemented in accordance with some embodiments of the disclosed subject matter. More particularly, FIG. 12 shows simulation data containing powerline noise from sample 0 to about sample 1250, a noise-free signal from about sample 1250 to about sample 3750, and additional powerline noise from about sample 3750 to about sample 5300, was recorded, and a QRS detection signal as an output. As shown in FIG. 12, techniques described herein correctly identified the QRS waveforms in both the low and high signal to noise ratio regions.

In some embodiments, efficiently implementing an S transform in hardware using the mechanisms described herein can facilitate many real-time applications, which may or may not be implemented in a remote monitor (i.e., more efficiently performing an S transform can be useful in applications without the power and size constraints imposed by a remote monitor form factor). For example, an S transform can be used in the analysis of magnetoencephalography data, which includes distinct temporal correlations in the brain that are not detectable using conventional. As another example, an S transform can be used for analysis of electroencephalogram (EEG) data of the brain, such as to analyze neural activity generally, for seizure detection, etc. Note that the range of frequencies, number of frequency bands, and resolution of frequency bands is generally application specific, and can be based on the expected components of the signal (e.g., based on the physics and/or physiology of how the signal is generated) and/or can be empirically determined.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, any other suitable media that is fleeting and devoid of any semblance of permanence during transmission, and/or any suitable intangible media.

It should be noted that, as used herein, the term mechanism can encompass hardware, software, firmware, or any suitable combination thereof.

It should be understood that the above described steps of the process of FIG. 7 can be executed or performed in any order or sequence not limited to the order and sequence shown and described in the figures. Also, some of the above steps of the process of FIG. 7 can be executed or performed substantially simultaneously where appropriate or in parallel to reduce latency and processing times.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways.

What is claimed is:

1. A method for finding a Stockwell transform of a signal, the method comprising:
receiving a plurality of samples of a signal generated by a sensor, wherein the plurality of samples includes at least:
i first samples of the signal from sample p−i to sample p−1, where i is an integer greater than one;
a sample p of the signal; and
i second samples of the signal from sample p+1 to sample p+i;
providing each of the plurality of samples to a first filter comprising 2i filter taps, wherein each filter tap of the 2i filter taps of the first filter corresponds to a component of a Stockwell transform windowing function for frequency band f₀, the output of each of the 2i filter taps corresponding to a multiplication of the sample and a coefficient based on the windowing function for frequency band f₀;
providing each of the plurality of samples to a corresponding filter tap of the 2i filter taps of the first filter;
generating a first component of a Stockwell transform of sample p for frequency band f₀ based on a sum of the outputs of the 2i filter taps of the first filter;
providing each of the plurality of samples to a second filter comprising 2i filter taps corresponding to components of a Stockwell transform windowing function for a second frequency band f₁; and
generating a second component of the Stockwell transform of sample p for frequency band f₁ based on a sum of outputs of the 2i filter taps of the second filter.

2. The method of claim 1, wherein providing the sample to a first filter comprising 2i filter taps comprises providing the sample to one or more logic blocks of a field programmable gate array, the one or more logic blocks of the field programmable gate array are configured to provide the 2i filter taps of the first filter.

3. The method of claim 1, further comprising receiving, from memory, a plurality of complex coefficients based on the windowing function for frequency band f₀, wherein each coefficient corresponds to a product of a windowing function and a Fourier kernel.

4. The method of claim 3, wherein the plurality of complex coefficients comprises a compact representation of the windowing function that includes only a subset of all coefficients needed to fully represent the entirety of the Fourier kernel are stored.

5. The method of claim 3, wherein the plurality of complex coefficients comprises a decimated subset of complex coefficients that provides an approximate representation of a full fidelity Fourier kernel of the Stockwell transform.

6. The method of claim 5, wherein the decimated subset is a truncation of the Fourier kernels of the Stockwell transform.

7. The method of claim 3, wherein the Stockwell transform for sample p and frequency band f₀ is characterized by
$$S [ p , {f}_{0} ) = {\sum }_{n = - i}^{i} x [ n ] \cdot w [ p - n , {f}_{0} ) \cdot {e}^{- j2 π {f}_{0} n}$$
where w[p−n, f)·e^{-j2πf}^{₀}ⁿ is the complex coefficient, and w[p−n, f) represents the windowing function and is characterized by
$$w [ p - n , {f}_{0} ) = \frac{\vert {f}_{0} \vert}{\sqrt{2 π}} \cdot {e}^{\frac{- ( {\vert {f}_{0} \vert ( p - n ) )}^{2}}{2}}$$
such that the complex coefficients for i is the complex conjugate of the complex coefficient for −i.

8. The method of claim 1, wherein the sensor comprises one or more electrocardiogram (ECG) leads, and wherein the signal is an ECG signal.

9. The method of claim 1, further comprising calculating a physiologic feature of the sample p based on the Stockwell transform for sample p.

10. The method of claim 9, wherein the physiologic feature is the Shannon energy of sample p.

11. The method of claim 1, further comprising providing the signal to an analog-to-digital converter that is configured to receive the signal and output the sample p at a predetermined sampling frequency.

12. A system for finding an Stockwell transform of a signal, the system comprising:
a sensor; and
a monitor comprising:
a processor coupled to the sensor, the processor programmed to:
receive a plurality of samples of the signal, wherein the plurality of samples includes at least:
i first samples of the signal from sample p−i to sample p−1, where i is an integer greater than one;
a sample p of the signal; and
i second samples of the signal from sample p+1 to sample p+i;
provide each of the plurality of samples to a first filter comprising 2i filter taps, wherein each filter tap of the 2i filter taps of the first filter corresponds to a component of a Stockwell transform windowing function for frequency band f₀, the output of each of the 2i filter taps corresponding to a multiplication of the sample and a coefficient based on the windowing function for frequency band f₀;
provide each of the plurality of samples to a corresponding filter tap of the 2i filter taps of the first filter;
generate a first component of a Stockwell transform of sample p for frequency band f₀ based on a sum of the outputs of the 2i filter taps of the first filter;
provide each of the plurality of samples to a second filter comprising 2i filter taps corresponding to components of a Stockwell transform windowing function for a second frequency band f₁; and
generate a second component of the Stockwell transform of sample p for frequency band f₁ based on a sum of outputs of the 2i filter taps of the second filter.

13. The system of claim 12, wherein the Stockwell transform is a discrete time Stockwell transform.

14. The system of claim 12, wherein the processor is further programmed to generate the first component of the Stockwell transform and the second component of the Stockwell transform after receiving sample p+i and prior to receiving sample p+(i+1).

15. The system of claim 12, further comprising memory storing a plurality of complex coefficients based on the windowing function for frequency band f₀, wherein each coefficient corresponds to a product of a windowing function and a Fourier kernel.

16. The system of claim 15, wherein the plurality of complex coefficients comprises a compact representation of the windowing function that includes only a subset of all coefficients needed to fully represent the entirety of the Fourier kernel are stored.

17. The system of claim 15, wherein the plurality of complex coefficients comprises a decimated subset of complex coefficients that provides an approximate representation of a full fidelity Fourier kernel.

18. The system of claim 17, wherein the decimated subset is a truncation of the Fourier kernels of the Stockwell transform.

19. The system of claim 15, wherein the processor comprises a field programmable gate array, and one or more logic blocks of the field programmable gate array are configured to provide the 2i filter taps of the first filter.

20. The system of claim 12, wherein the Stockwell transform for sample p and frequency band f₀ is characterized by
$$S [ p , {f}_{0} ) = {\sum }_{n = - i}^{i} x [ n ] \cdot w [ p - n , {f}_{0} ) \cdot {e}^{- j2 π {f}_{0} n}$$
where w[p−n, f)·e^{-j7πf}^{₀}ⁿ is the complex coefficient, and w[p−n, f) represents the windowing function and is characterized by
$$w [ p - n , {f}_{0} ) = \frac{\vert {f}_{0} \vert}{\sqrt{2 π}} \cdot {e}^{\frac{- ( {\vert {f}_{0} \vert ( p - n ) )}^{2}}{2}}$$
such that the complex coefficients for i is the complex conjugate of the complex coefficient for −i.

21. The system of claim 12, wherein the Stockwell transform for sample p has a complexity of O(NM) where N is 2i+1, and M is a number of frequency bands f₀ to fₘ analyzed to perform the Stockwell transform, wherein the system comprises one filter for each frequency band.

22. The system of claim 12, further comprising an analog-to-digital converter that is configured to receive the signal and output the sample p at a predetermined sampling frequency.

23. The system of claim 12, wherein the sensor comprises one or more electrocardiogram (ECG) leads, and wherein the signal is an ECG signal.

24. The system of claim 12, wherein the processor is further configured to calculate the Shannon energy of sample p based on the Stockwell transform for sample p.

25. A system for monitoring and providing feedback about a physiological condition of a subject, the system comprising:
a sensor configured to monitor at least one condition of the subject and generate physiological feedback data;
a processor configured to receive the physiological feedback data from the sensor and programmed to:
implement a filter bank with a predetermined number of coefficients and taps selected to perform a Stockwell transform on the physiological feedback data and provide a frequency domain data of the physiological feedback data;
analyze the frequency domain data using a physiological monitoring criteria;
generate a report about the physiological condition of the subject based on the analysis of the frequency domain data using the physiological monitoring criteria;
a display configured to display the report about the physiological condition of the subject.

26. The system of claim 25, wherein the Stockwell transform is a discrete time Stockwell transform (DTST).

27. The system of claim 25, wherein the processor comprises a field programmable gate array, and one or more logic blocks of the field programmable gate array are configured to provide filter bank.

28. The system of claim 25, wherein the filter bank performs the Stockwell transform entirely in the time domain.
